(19) United States
(12) Patent Application Publication
(10) Pub. No.: US 2002/0049350 A1
(43) Pub. Date: Apr. 25, 2002

(54) Process for preparing thrombin receptor antagonist building blocks

(76) Inventors: Xiaoyong Fu, Edison, NJ (US); Chou-Hong Tann, Berkeley Heights, NJ (US); T.K. Thiruvengadam, Kendall Park, NJ (US); Jianguo Yin, Edison, NJ (US); Timothy L. McAllister, Westfield, NJ (US); Cesar Colon, Rio Piedras, PR (US)

Correspondence Address: SCHERING-PLOUGH CORPORATION, PATENT DEPARTMENT (K-6-1, 1990), 2000 GALLOPING HILL ROAD, KENILWORTH, NJ, 07033-0530, US

(21) Appl. No.: 09/891,601

(22) Filed: Jun. 26, 2001

Related U.S. Application Data
(60) Provisional application No. 60/214,581, filed on Jun. 28, 2000.

(51) Int. Cl. C07C 69/73

(52) U.S. Cl. 560/60; 560/183

(57) ABSTRACT

A single batch process for preparing (R)-benzyl-4-hydroxy-2-pentynoate by reacting (R)-3-butyn-2-ol with 1,1,1,3,3,3-hexamethyldisilazane to silylate the starting alcohol, followed by deprotonation with an alkyl lithium compound, then a reaction with a haloformate compound, and finally a hydrolysis reaction to arrive at the product ester.

### BACKGROUND OF THE INVENTION

1. Field of the Invention

The invention relates to a process for preparing a building block for thrombin receptor antagonists, and more particularly, a process for producing (R or S)-benzyl-4-hydroxy-2-pentynoate. 2. Description of the Related Art

Thrombin receptor antagonists have been disclosed as being particularly potent therapeutic agents in many applications. See e.g., U.S. Pat. No. 6,063,847; WO 94/03479; and Bematowicz et al., J. Med. Chem., 39, pp. 4879-4887 (1996). They have been utilized in the treatment of thrombotic, inflammatory, atherosclerotic and fibroproliferative disorders, as well as other disorders in which thrombin and its receptor play a pathological role.

One-pot procedures for two or more sequential reactions are of great importance in organic synthesis, both in laboratories and industrial production. See e.g., Misner et al., Org. Proc. Res. Develop, 1:77 (1997). The syntheses currently employed for producing building blocks for thrombin receptor antagonists involve complex multi-step processes. See e.g., U.S. Pat. No. 6,063,847. Tetrahydropyranyl (THP) has been used as a protecting group in such types of syntheses. THP protecting groups, however, are problematic for commercial applications. Syntheses involving THP are not very suitable for scale-up operations due to several drawbacks, especially the requirement of having to utilize multiple-step operations that need to have intermediate compounds isolated between steps. Other common disadvantages include the necessity of carrying out lithiation steps under low operating temperatures (e.g., −78° C.), difficult filtration of resins in deprotonation steps, required use of chromatographic columns and overall yields which are moderate to poor. See id.

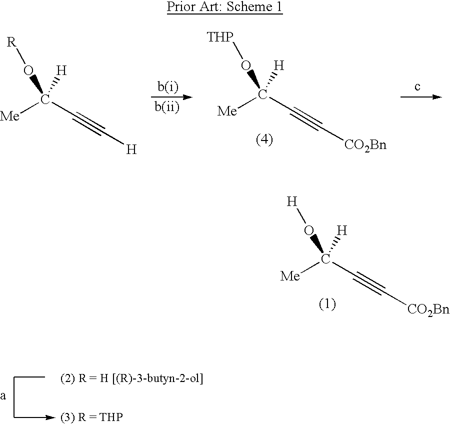

Key:

Me=methyl group;

CO₂Bn=benzyl formate group

THP=tetrahydopyranyl group

(a) dihydropyran, PTSA (para-toluene sulfonic acid), THF (tetra-hydrofuran), 0° C. to room temperature;

(b) (i) n-BuLi (n-butyl lithium), THF, −78° C.;

(ii) ClCO₂Bn (benzyl chloroformate), −78° C. to room temperature;

and

(c) DOWEX 50WX8-100 resin, MeOH (methanol), room temperature.

In synthesizing thrombin receptor antagonist building blocks (e.g., (R)-benzyl-4-hydroxy-2-pentynoate), it would be beneficial if an efficient, single batch scalable preparation could be accomplished.

It is an object of the invention to provide a process for synthesizing thrombin receptor antagonist building blocks, which overcome the drawbacks of prior art processes.

It is a further object of the invention to provide syntheses for thrombin receptor antagonist building blocks that can be carried out efficiently and economically in a single batch process.

It is yet another object of the invention to provide syntheses for thrombin receptor antagonist building blocks that can be carried out at a variety of temperatures, even up to room temperature, and still return good to excellent product yields.

It is still a further object to provide stable, inexpensive and efficient protecting groups, which can be employed during the syntheses of thrombin receptor antagonist building blocks.

These and other objects of the invention will become apparent as the description progresses.

Certain aspects of the inventors' work have been disclosed in Gaifa Lai et al., Synthetic Communications, A One-Pot and Efficient Preparation of (S)-Benzyl-4-Hydroxy-2-Pentynoate From (S)-3-Butyn-2-ol, Vol. 29(17), pp. 301 1 -3016 (1999).

### DEFINITIONS AND USAGE OF TERMS

The term “alkyl,” as used herein, means an unsubstituted or substituted, straight or branched, hydrocarbon chain, preferably having from one to twelve carbon atoms.

The term “cycloalkyl,” as used herein, means an unsubstituted or substituted, saturated carbocyclic ring, preferably having from three to eight carbon atoms.

The term “alkenyl,” as used herein, means an unsubstituted or substituted, unsaturated, straight or branched, hydrocarbon chain having at least one double bond present, preferably having from one to twelve carbon atoms.

The term “aryl,” as used herein, means a substituted or unsubstituted, aromatic carbocyclic ring. Preferred aryl groups include phenyl, tolyl, xylyl, cumenyl and napthyl.

The term “aralkyl,” as used herein, means an alkyl moiety substituted with an aryl group. Preferred aralkyls include benzyl, phenylethyl and 1- and 2-naphthylmethyl.

It is understood by those skilled in the art that the chiral compounds described herein exist in both (R) and (S) configurations. (S) refers to the counterclockwise arrangement of the high to low priority substituents about the asymmetric carbon atom. (R) refers to the clockwise arrangement of the high to low priority substituents about the asymmetric carbon atom. The (R) configuration chiral compound is specifically described herein. However, it is known to those skilled in the art that the (S) configurations can also be produced from appropriately configured starting materials.

### SUMMARY OF THE INVENTION

The present invention relates to a process for preparing thrombin receptor antagonist building blocks. More specifically, the invention is directed to a process for producing a (R) or (S) enantiomer of a compound having the formula (I):

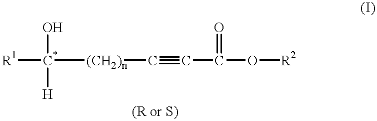

where,

R¹ is a substituted or unsubstituted, alkyl, cycloalkyl, alkenyl, aryl or aralkyl group,

R² is a substituted or unsubstituted, alkyl, cycloalkyl, alkenyl, aryl or aralkyl group, and

n is a number from 0 to 12.

The inventive process comprises:

(a) silylating a compound having the formula (II):

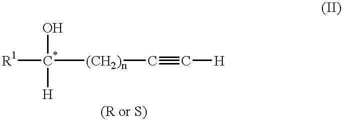

where

R¹ and n are defined the same as above,

to form an intermediate compound having the formula (III):

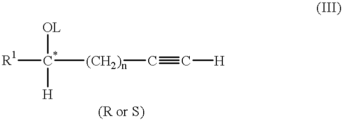

where

R¹ and n are defined the same as above and

L is a silyl protecting group,

(b) deprotonating the intermediate compound of formula (III) and reacting it with a haloformate compound having the formula (IV):

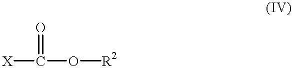

where

R² is defined the same as above and

X is a halogen atom,

to form a compound having the formula (V):

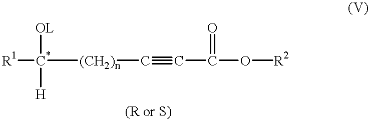

where

R¹, R², n and L are defined the same as above, and

(c) replacing the L group with a hydrogen atom (H) in the compound of formula (V) via a hydrolysis reaction or a reaction with an anhydrous acidic medium to form the compound of formula (I).

The preferable and most commercially viable enantiomer produced by this invention is the one with a (R) configuration for the compound of formula (I), which can be used as a building block for making a biologically active thrombin receptor antagonist. It is in this area that the invention should have important commercial benefits. In addition, the invention provides a viable method for making (S) enantiomers. When a structural formula herein depicts a (R) or (S) enantiomer, it is understood that the corresponding enantiomer can also be prepared by the same method if one starts with the corresponding desired configuration for the starting material.

Surprisingly, the inventive process can be carried out in a single batch, thus allowing for an efficient and economically feasible one-pot process to prepare thrombin receptor antagonist building blocks.

A further understanding of the invention will be had from the following description of the preferred embodiments.

### DETAILED DESCRIPTION OF THE INVENTION

In the compound of the formula (I):

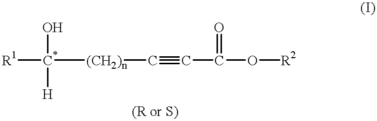

where

R¹, R² and n are the same as defined above.

The R¹ group is preferably alkyl, most preferably, methyl. As to the R² group, it is preferably substituted or unsubstituted, alkyl, aryl or aralkyl. The alkyl group for R² may be a straight-chain alkyl group, but is preferably branched, such as a tert-butyl group. The aralkyl group for R² is preferably unsubstituted, for example, a benzyl group. For the methylene chain off of the asymmetric carbon ((CH₂)ₙ), there may be up to twelve methylene groups present, but preferably, no groups are present (n=0).

A preferred silylating agent is 1,1,1,3,3,3-hexamethyldisilazane (HMDS):

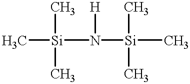

which is useful for silylating a compound having the formula (II) in step (a) of the process:

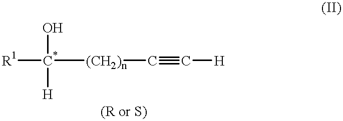

where

R¹ and n are defined the same as above,

The use of HMDS as a silylating agent will advantageously provide trimethylsilyl (TMS) as a silyl protecting group (L) in a resultant intermediate compound having the formula (III):

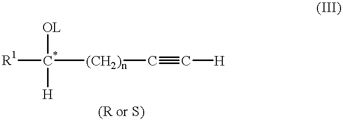

where

R¹ and n are defined the same as above and

L is a silyl protecting group.

Preferably, L is a trialkylsilyl group. Most preferably, as is given when HMDS is the silylating agent, L is a trimethylsilyl (TMS) group, as shown in the compound of formula (IIIA):

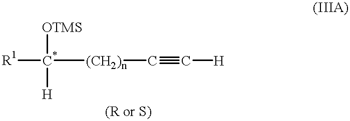

where

R¹ and n are defined the same as above.

In deprotonating the intermediate compound of formula (III) or formula (IIIA) in step (b) of the process:

[chemical structure]

where

R¹, n and L are defined the same as above,

a carboanion intermediate compound of the formula (IIIB) is formed. A deprotonating agent, such as an organometallic reagent, R⁴M or R⁴MX, where R⁴ is a hydrocarbon group, M is a metal and X is a halogen atom, is advantageously utilized to form the carboanion. Preferred deprotonating agents include a lithiating agent, R⁴Li, and a Grignard agent, R⁴MgX. For example, an alkyl lithium compound may be used to deprotonate the subject compound to form an intermediate compound having the formula (IIIC.1):

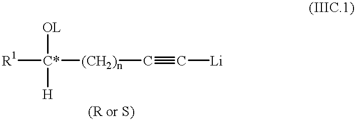

where

R¹, n and L are defined the same as above,

Similarly, Grignard reagents will deprotonate the subject compound and form an intermediate compound having the formula (IIIC.2):

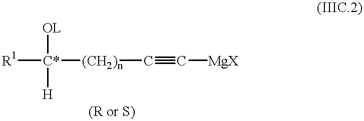

where

R¹, n and L are defined the same as above,

Preferably, a suitable organometallic reagent is employed as a solution in an inert solvent. This solution is advantageously added under an inert atmosphere, such as nitrogen, to effect the deprotonation step. Other suitable organometallic reagents are known in the art and are commercially available or may be prepared from alkyl, cycloalkyl, aryl or aralkyl halides and the like by conventional methods in the art. The preferred organometallic reagents are organolithium and organomagnesium (Grignard) reagents, Most preferably, alkyl lithium compounds, especially n-butyl lithium, and alkylmagnesium chlorides or bromides are utilized to deprotonate the intermediate compound of formula (III) or formula (IIIA).

The preferred inert (i.e., non-reactive) solvents include tetrahydrofuran (THF), diethyl ether, tert-butylmethylether, dimethoxyethane, dimethoxymethane, toluene, hexane, heptane or a mixture thereof. The most preferred solvent is THF.

The deprotonated intermediate compound (e.g., a compound of the formula (IIIC.1) or (IIIC.2)) is advantageously reacted with a haloformate compound having the formula (IV):

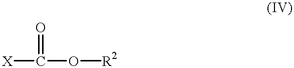

where R² is defined the same as above and

X is a halogen atom, preferably, a chlorine or bromine atom, most preferably, a chlorine atom.

This reaction results in a compound having the formula (V):

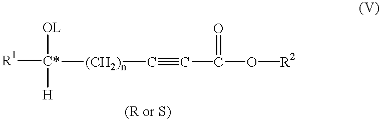

where

R¹, R², n and L are defined the same as above.

Advantageously, the next step in the process is to hydrolyze the compound of formula (V) to form the desired product compound of the formula (I). The hydrolysis reaction is best carried out in an acidic medium, such as aqueous sulfuric acid. Other possible acidic mediums include aqueous nitric acid and typical aqueous weak acids, such as aqueous acetic acid.

Alternatively, the L group in formula (V) may be replaced with a hydrogen atom via a non-aqueous acidic reaction with an acidic reagent, such as gaseous hydrogen chloride (HCI) or a mixture of thionyl chloride (SOCl₂) in methanol (CH₃OH), which generates dry (anhydrous) hydrogen chloride (HCl). Though less preferred than hydrolysis (for economic feasibility reasons), these and other like anhydrous reactions will provide the same product as hydrolysis.

Enantiomerically pure (R)-3-butyn-2-ol (formula (II), where R¹ =methyl and n=0) is a starting material which may be obtained commercially from DSM Fine Chemicals, Inc. (Saddle Brook, N.J.) or by resolution of the corresponding racemic mixture through procedures known in the art. Trimethylsilyl is a preferred silyl protecting group (L=TMS) in view of its facile introduction and removal under mild conditions. See, Greene, TW, et al., 1991, Protective Groups in Organic Chemistry, 2d Ed., J. Wiley & Sons, Inc., N.Y.

The inventive process provides a novel, one-pot procedure for efficiently preparing (R or S)-benzyl-4-hydroxy-2-pentynoate (compound 1)) from (R or S)-3-butyn-2-ol (compound (2)) via a lithiation reaction with n-butyl lithium (n-BuLi). Preferably, an exact or near-exact calculated amount, based on equivalents or moles, of the lithiating agent is employed.

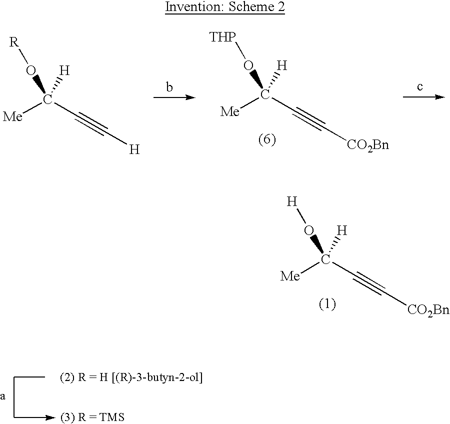

Key:

Me=methyl group

CO₂Bn=benzyl formate group

TMS=trimethyl silyl protecting group

HMDS=1,1,1,3,3,3-hexamethyldisilazane

(a) HMDS (0.65 equivalents), THF (tetrahydrofuran), 68-70° C.;

(b) (i) n-BuLi (n-butyl lithium), −30 to −25° C. and

(ii) ClCO₂Bn (benzyl chloroformate), −30 to −25° C.; and

(c) 6 N H₂SO₄ (aqueous sulfuric acid).

As depicted in Scheme 2, the silylation of (R)-3-butyn-2-ol (compound (2)) can be accomplished with the addition of about 0.65 equivalents of HMDS (step (a)) in THF at 68-70° C. for about 2 hours. This reaction cleanly affords the silyl ether (compound (5)) in near quantitative yields (as monitored by ¹HNMR) with a concomitant release of ammonia as a byproduct. It was found that a mixture of solvent with the starting alcohol (compound (2)) can be advantageously adjusted to an acidic level with the addition of sulfuric acid, preferably, to a pH of approximately from 3 to 4, before addition of the HMDS in order to facilitate the silylation reaction. Silylation can sometimes be slow when the mixture of solvent and the starting alcohol (compound (2)) is neutral or slightly basic, for example, an approximate pH of from 7 to 8. When the reaction is complete, the mixture can be cooled down for direct use in the next step or the product may first be purified by distillation or other known methods before the next step is commenced.

The conversion in step (b) of the silyl ether with an acetylenic hydrogen (compound (5)) to a silyl ether with a benzyl formate substituent (compound (6)) advantageously proceeds through a two-step sequence consisting of deprotonation, such as is provided by lithiation with n-butyl lithium (step (b)(i)), followed by a carbobenzyloxylation reaction with benzyl chloroformate (step (b)(ii). This method provides high yields of the desired ester. Other advantages of this protocol include the use of economically efficient reagents and ease of operation on a large scale.

Finally, the removal of the TMS protecting group in compound (6) can be readily effected in step (c) by direct treatment with an aqueous acid, such as a 6 N H₂SO₄ solution, which provides the desired product (compound (1)) in about a 86% overall yield starting from the alcohol of compound (2).

Preferable silylating agents for step (a) include HMDS, BSA (bis-trimethylsilyl acetamide), BSU (bis-trimethylsilyl urea), TMS-Cl (trimethyl silyl chloride), TES-Cl (triethyl silyl chloride) and TBDMS-Cl (tert-butyl dimethyl silyl chloride). The optimum amount of silylating agent used in step (a) can be easily determined according to known stoichiometric principals. For instance, at least about 0.5 equivalents should be used for the HMDS, BSA and BSU silylating agents (because there are two silicon atoms), while twice that amount, or at least about 1 equivalent, is best used for the TMS-Cl, TES-Cl and TBDMS-Cl silylating agents (which have only one silicon atom).

Efficient deprotonating agents for step (b)(i) include lithiating agents, such as n-butyl lithium (n-BuLi), lithium hexamethyldisilylazide (LHMDS) and lithium diisopropylamide (LDA). As disclosed above, Grignard (R⁴MgX) and other organometallic (R⁴M) reagents are also suitable deprotonating agents. The preferred amount of deprotonating agent to be added in step (b)(i) is an exact or near-exact equivalent ratio (ie., approximately between 0.9 and 1 equivalents). Exact and near-exact loads of a deprotonating agent should provide the best results and minimize processing problems. Higher loads will also work, though they may be less efficient and/or less process friendly. In addition, higher loads will likely result in lower yields and/or less pure final products.

Preferable haloformate compounds to be utilized in step (b)(ii) include benzyl chloroformate, benzyl bromoformate and tert-butyl chloroformate. The most preferable haloformate compound is benzyl chloroformate. The preferred amount of haloformate compound to be added in step (b)(ii) is from about 1 to about 1.5 equivalents, most preferably, from about 1 to about 1.2 equivalents

The inventive process can prepare a (R) or (S) enantiomer of a compound having the formula (I):

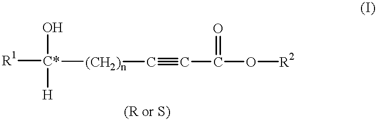

where,

R¹ is a substituted or unsubstituted, alkyl, cycloalkyl, alkenyl, aryl or aralkyl group,

R² is a substituted or unsubstituted, alkyl, cycloalkyl, alkenyl, aryl or aralkyl group, and

n is a number from 0 to 12.

The process includes:

(a) silylation of a compound having the formula (II):

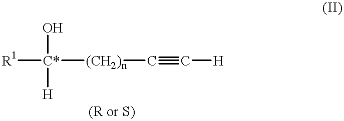

where

R¹ and n are defined the same as above,

which results in an intermediate compound having the formula (III):

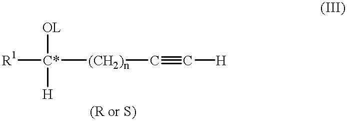

where

R¹ and n are defined the same as above and

L is a silyl protecting group,

(b) deprotonation of the intermediate compound of formula (III) followed by reaction with a haloformate compound having the formula (IV):

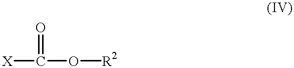

where

R² is defined the same as above and

X is a halogen atom,

which results in a compound having the formula (V):

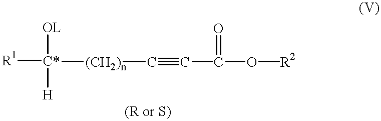

where

R¹, R², n and L are defined the same as above, and

(c) reaction of the compound of formula (V) with an aqueous or anhydrous acidic medium to form the compound of formula (I).

A particularly preferred embodiment of the invention is a batch process for preparing a compound having the formula (I.1):

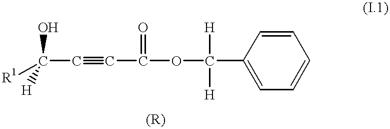

where,

R¹ is a substituted or unsubstituted, alkyl, cycloalkyl, alkenyl, aryl or aralkyl group,

The process comprises:

(a) reacting a compound having the formula (II.1) with a silylating agent (LA) to form an intermediate compound having the formula (III.1):

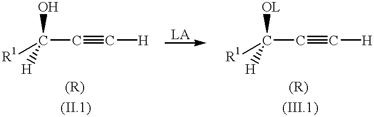

where

R¹ is defined the same as above and

L is a silyl protecting group,

(b) deprotonating the intermediate compound of formula (III.1) and reacting it with a benzyl haloformate compound having the formula (IV.1) to form a compound having the formula (V.1):

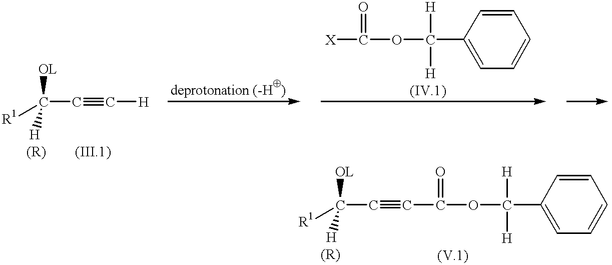

where

R¹ and L are defined the same as above, and

X is a chlorine or bromine atom,

(c) hydrolyzing the compound of formula (V.1) to form the compound of formula (I.1).

When R¹ is a methyl group, the main starting material (compound of the formula (II.1)) is (R)-3-butyn-2-ol (compound (2) in Scheme 2) and the product obtained (compound of the formula (I.1)) is (R)-benzyl-4-hydroxy-2-pentynoate (compound (1) in Scheme 2). A single batch process for preparing this product is highly efficient and economical.

The following non-limiting Examples will help illustrate the practice of the invention. The experiments show the effects of certain processing parameters: a) order of addition of the reactants with a change of temperature and b) nature of the deprotonating/lithiating ligand.

### Example 1

### n-BuLi (n-butyl lithium) as Lithiating Agent

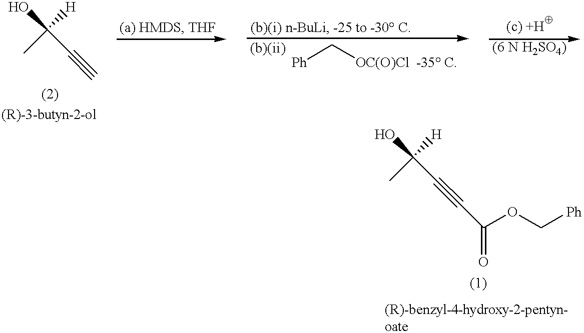

Key:

HMDS=1,1,1,3,3,3-hexamethyldisilazane

n-BuLi=n-butyl lithium

THF=tetrahydrofuran

Ph=phenyl group

Silylation and Deprotonation

STEP (a): To a solution of 7.5 g (107 mmol) of (R)-3-butyn-2-ol (compound (2)) and 15 ml (0.65 equivalents) of 1,1,1,3,3,3-hexamethyldisilazane (HMDS) in 30 ml of THF was added 2 drops of concentrated sulfuric acid. The solution was heated to reflux for 1 hour.

H¹-NMR analysis indicated that the protection of the hydroxyl group was completed: [H¹-NMR (400 MHz, CDCl₃): 4.52 (1H, dq),2.40 (1H, d),1.45 (3H, d),0.18 (9H, s)].

The resultant silylated butynol was distilled out in THF by heating the solution to 120 ° C. The residue was mixed with 10 ml of toluene and a majority of the solution was distilled out again. The combined distillate was then mixed with 200 ml of THF and the resulting solution was cooled to −30 ° C.

STEP (b)(i): Then, n-butyl lithium (n-BuLi) in hexane (86 mmol, 0.80 equivalents) was charged dropwise over 30 minutes while the temperature was maintained at −30 ° C. A small amount of reaction mixture (˜2 to 3 drops) was quenched into 1 ml of CD₃COOD and the mixture was checked by ¹H NMR.

It was important to carry out the sampling under nitrogen. The disappearance of the doublet at 2.2 ppm (the proton on the acetylene carbon) meant a complete reaction has occurred. Depending on the ratio of the doublet at 2.2 ppm to the multiple at 4.3 ppm (the proton at the hydroxyl carbon), more n-butyl lithium was added during the reaction. A ratio of 0.2 to 1, for example, indicated that about 20% of starting material was still present and an additional amount of n-butyl lithium (17 mmol, 20% of the amount initially charged) was added. The same monitoring procedure was repeated until the deprotonated acetylene was >97%. This multiple sampling procedure ensured that 1 equivalent of n-butyl lithium was charged with >97% accuracy, regardless of different moisture levels in different experiments and different n-butyl lithium concentrations. The reaction mixture comprising a lithium acetylide solution was kept at <-25 ° C. and used immediately in the next step.

(i) Reverse Addition

Lithiated Acetylide Solution Charged Into Benzyl Chloroformate Solution.

Coupling with Carbobenzylate Compound—Reverse Addition

STEP (b)(ii): To a solution of benzyl chloroformate (139 mmol, 1.3 equivalents) in 50 ml of THF at −35 ° C. was transferred slowly (30 min) through a cannula the lithium acetylide solution prepared in step (b)(i) above.

STEP (c): The reaction mixture was stirred for another 30 min at −25 ° C. and quenched with 50 ml of 6N H₂SO₄ solution. The mixture was stirred for about one hour and the organic phase was separated and washed with 5% of ammonium chloride and then water. Solvent was removed under vacuum to give 30.6 g of red oil. The yield of the desired product (compound (1)) was determined by HPLC as 18.6 g, a 86% yield.

H¹-NMR (400 MHz, CDCl3): 7.40 (5H, m), 5.23 (2H, s), 4.65 (1H, q, J=6.7 Hz), 2.10 (1H, br. s), 1.53 (3H, d, J=6.7 Hz).

C¹³-NMR (100.6 MHz, CDCl3): 153.2,134.6,128.7,128.6,88.8, 75.6, 67.8, 58.0, 23.2.

Under-Charge versus Over-Charge of n-BuLi

It is important to utilize the sampling procedure described above to monitor the deprotonation/lithiation reaction. Both over-charge and under-charge of n-BuLi could cause a significant reduction in the yield. As indicated by multiple experiments, when n-BuLi was 10% under-charged, the isolated yield was about 75%. On the other hand, the yield dropped to about 65% when a 10% over-charge of n-BuLi was added.

Conclusion

Amount of Deprotonating/Lithiating Agent

To maximize product yields, it is preferable to use an exact or near-exact equivalent amount of n-BuLi as the deprotonating/lithiating agent.

(ii) Normal Addition

Benzyl chloroformate solution charged into lithiated acetylide solution

Coupling with Carbobenzylate Compound—Normal Addition

The normal addition method requires low temperature operation for the coupling reaction, as is exemplified by the following two experiments:

A. at −65 ° C. (lower temperature): 3.73 g, (53.3 mmol) of (R)-3-butyn-2-ol (compound (2)) was silylated (TP (a) and deprotonated (STEP (b)(i)) in the same way as described above. The solution was then cooled to −75 ° C. STEP (b)(ii.): Benzyl chloroformate (11.8 g, 63.9 mmol, 1.2 equivalents) was slowly charged into the solution and the temperature was maintained below −65 ° C. The reaction mixture was then warmed to −30 ° C. in about 2 hours. The mixture was then treated with an acidic medium (STEP (c)) in the same way as described above. The final solution contained 10.8 g of product (compound (1)), a 85% yield.

B. at −30 ° C. (higher temperature): STEP b(ii): Benzyl chloroformate (11.8 g, 63.9 mmol, 1.2 equivalents) was charged at −30 ° C. to a lithium acetylide solution made with 3.73 g of (R)-3-butyn-2-ol (compound (2)). The mixture was subjected to the same work-up procedure (STEP (c): addition of sulfuric acid) as described above. 3.4 g of the desired product (compound (1)) was recovered, a 32% yield.

Conclusion

### Effect of Order of Addition of Reactants and Temperature

For step (b)(ii) of the process, it is less preferred to charge the carbobenzylated compound into the deprotonated/lithiated acetylide solution (Normal Addition), because that order of addition requires a lower temperature to return decent yields. Rather, it is best to charge the deprotonated/lithiated acetylide solution into the carbobenylated compound (Reverse Addition), since this order of addition provides good yields at both low and high temperatures.

### Example 2

### LDA as Lithiating Agent—Normal Addition Method at −65° C.

STEP (a): 1,1,1,3,3,3-hexamethyldisilazane (HMDS) (8.9 mL, 41.7 mmole) was added slowly to a solution of 6 ml (76.6 mmole) of (R)-3-butyn-2-ol (compound (2)) and 50 ml tetrahydrofuran in a 250 ml three-necked round bottom flask equipped with a nitrogen inlet, thermometer and reflux condenser. The mixture was agitated for 13 hours at room temperature.

STEP (b)(i): The solution was cooled to −78° C. with a dry ice/acetone bath. Lithium diisopropylamide (LDA) (40 mL of 2M solution in heptane/THF/ethylbenzene, 80 mmole) was charged dropwise to maintain the reaction temperature below −67° C.

STEP (b)(ii): After agitation of the cold mixture for 30 min, benzyl chloroformate (11.0 mL, 77.0 mmole, ˜1 equivalent) was slowly added to keep the temperature below −65° C.

STEP (c): The reaction mixture was stirred for an additional 30 min before it was quenched by a slow addition of 60 ml 2N aqueous H₂SO₄. The resultant two-layer mixture was agitated for about 1 hour while letting the mixture warm to room temperature and the two layers were separated. The organic layer was washed with aqueous NaHCO₃ and then water and dried over Na₂SO₄. The solvent was removed under vacuum to provide a brown thick oil (15.1 g). The crude oil was purified by column chromatography (silica gel, 20% EtOAc/Hexane) to provide 12.3 g of product (compound 1)), a 80% yield.

Conclusion

n-BuLi Versus LDA as Deprotonating/Lithiating Agent

Both n-BuLi and LDA are efficient deprotonating/lithiating agents for the Normal Addition method at lower temperatures. (Both would also be excellent lithiating candidates for the deprotonating step at higher temperatures if the Reverse Addition method were used.)

It was surprising that the invention disclosed herein provides an efficient and economical way for synthesizing thrombin receptor antagonist building blocks. It was further surprising that TMS would exhibit such high stability and protecting characteristics. Moreover, it was unexpected that the reactions were effective at higher temperatures, even as high as room temperature, for the reverse addition method.

The above description is not intended to detail all modifications and variations of the invention, which will become apparent to the skilled worker upon reading the description. It is intended, however, that all obvious modifications and variations be included within the scope of the present invention, which is defined by the following claims.

What is claimed is:

1. A process for preparing a (R) or (S) enantiomer of a compound having the formula (I): 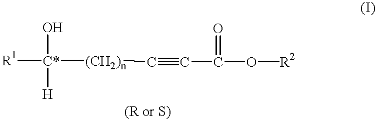
where,
R¹ is a substituted or unsubstituted, alkyl, cycloalkyl, alkenyl, aryl or aralkyl group,
R² is a substituted or unsubstituted, alkyl, cycloalkyl, alkenyl, aryl or aralkyl group, and
n is a number from 0 to 12,
the process comprising:
(a) silylating a compound having the formula (II): 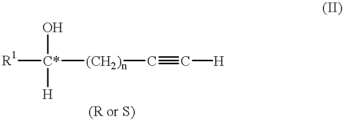
where
R¹ and n are defined the same as above, to form an intermediate compound having the formula (III): 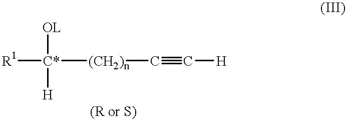
where
R¹ and n are defined the same as above and
L is a silyl group,
(b) deprotonating the intermediate compound of formula (III) and reacting it with a haloformate compound having the formula (IV): 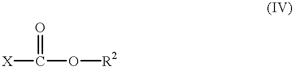
where
R² is defined the same as above and
X is a halogen atom, to form a compound having the formula (V): 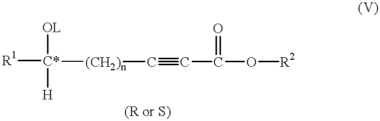
where
R¹, R², n and L are defined the same as above, and
(c) hydrolyzing the compound of formula (V) to form the compound of formula (I).

2. The process according to claim 1, wherein the (R) enantiomer of the compound of formula (I) is produced.

3. The process according to claim 2, where R¹ is the alkyl group.

4. The process according to claim 3, where R¹ is a methyl group.

5. The process according to claim 2, where R² is a substituted or unsubstituted, branched or straight-chain alkyl group or a substituted or unsubstituted aryl or aralkyl group.

6. The process according to claim 5, where R² is a tert-butyl or benzyl group.

7. The process according to claim 6, where R² is the benzyl group.

8. The process according to claim 2, which is carried out in a single batch.

9. The process according to claim 2, where L is a trialkylsilyl group.

10. The process according to claim 9, where L is a trimethylsilyl group.

11. The process according to claim 2, wherein the deprotonation of step (b) is carried out in the presence of a lithiating agent or a Grignard reagent.

12. The process according to claim 11, wherein the lithiating agent is an alkyl lithium compound.

13. The process according to claim 12, wherein the alkyl lithium compound is n-butyl lithium or lithium diisopropylamide.

14. The process according to claim 2, wherein the halogen atom in the haloformate compound is chlorine or bromine.

15. The process according to claim 2, wherein the hydrolysis of step (c) is carried out in an acidic medium.

16. A process for preparing a (R) or (S) enantiomer of a compound having the formula (I): 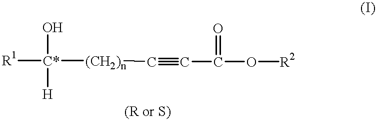
where,
R¹ is a substituted or unsubstituted, alkyl, cycloalkyl, alkenyl, aryl or aralkyl group,
R² is a substituted or unsubstituted, alkyl, cycloalkyl, alkenyl, aryl or aralkyl group, and
n is a number from 0 to 12,
the process comprising:
(a) silylating a compound having the formula (II): 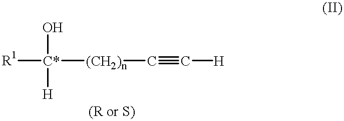
where
R¹ and n are defined the same as above, to form an intermediate compound having the formula (III): 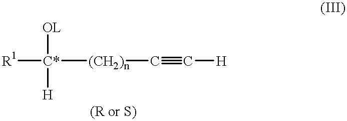
where
R¹ and n are defined the same as above and
L is a silyl group,
(b) deprotonating the intermediate compound of formula (III) and reacting it with a haloformate compound having the formula (IV): 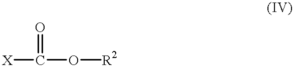
where
R² is defined the same as above and
X is a halogen atom, to form a compound having the formula (V): 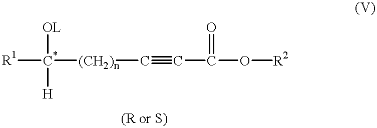
where
R¹, R², n and L are defined the same as above, and
(c) reacting the compound of formula (V) with a hydrous or anhydrous acidic medium to form the compound of formula (I).

17. A batch process for preparing a compound having the formula (I.1): 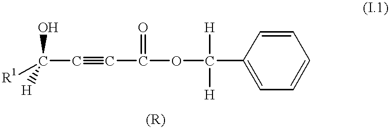
where,
R¹ is a substituted or unsubstituted, alkyl, cycloalkyl, alkenyl, aryl or aralkyl group,
the process comprising:
(a) reacting a compound having the formula (II.1) with a silylating agent: 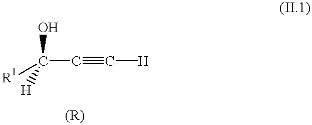
where
R¹ is defined the same as above, to form an intermediate compound having the formula (III.1): 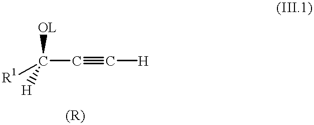
where
R¹ is defined the same as above and
L is a silyl group,
(b) deprotonating the intermediate compound of formula (III.1) and reacting it with a haloformate compound having the formula (IV.1): 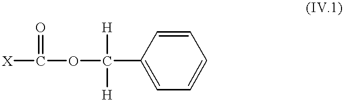
where
X is a chlorine or bromine atom, to form a compound having the formula (V.1): 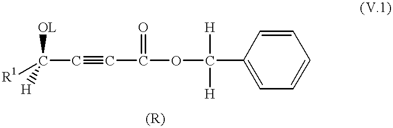
where
R¹ is defined the same as above, and
(c) hydrolyzing the compound of formula (V.1) to form the compound of formula (I.1).

18. The process according to claim 17, wherein the silylating agent is 1,1,1,3,3,3-hexamethyldisilazane.

19. The process according to claim 18, which is carried out in a single batch.

20. The process according to claim 18, wherein R¹ is a methyl group.
